(19) United States
(12) Patent Application Publication
(10) Pub. No.: US 2019/0015022 A1
(43) Pub. Date: Jan. 17, 2019

(54) CONTACT LENS FOR DETECTING GLUCOSE LEVEL IN TEARS AND METHOD FOR MAKING THE SAME

(71) Applicant: HON HAI PRECISION INDUSTRY CO., LTD., New Taipei (TW)

(72) Inventor: HSIU-WEN CHIEN, New Taipei (TW)

(21) Appl. No.: 15/658,483

(22) Filed: Jul. 25, 2017

(30) Foreign Application Priority Data
Jul. 11, 2017 (TW) 106123257

(51) Int. Cl. A61B 5/145; A61B 5/1486; A61B 5/00; G02C 11/00; G02B 1/04; B29D 11/00

(52) U.S. Cl. CPC A61B 5/14532; A61B 5/1486; A61B 5/6821; G02C 11/10; G02B 1/043; B29K 2105/0002; B29D 11/00134; A61B 2562/0285; A61B 2562/125; G02B 2207/101; B29D 11/00038

(57) ABSTRACT

A contact lens for detecting body glucose in tears includes a gel substrate, a plurality of platinum nanoparticles dispersed in the gel substrate, and a biosensor received in the gel substrate. The biosensor receives the glucose from tears as glucose oxidase and the glucose acid and hydrogen peroxide (H₂O₂) produced are decomposed to water and hydrogen peroxide. The platinum nanoparticles function as a catalytic agent to accelerate the degradation of the hydrogen peroxide into water and oxygen, avoiding or reducing hypoxia of the eye.

### FIELD

The subject matter herein generally relates to health, and more particularly, to a contact lens for detecting body glucose level by reference to tears and a method for making the contact lens.

### BACKGROUND

Monitoring glucose levels in blood regularly is very important in diabetes management. Body fluids, including tears, are potential sources for tracking glucose levels. Tracking glucose levels in tears is non-invasive and convenient. Currently, some available contact lenses can monitor the glucose levels in tears. However, a user who wears these glucose monitoring contact lens for a long time may suffer from hypoxia, and hypoxia may cause eye damage such as acute red eye, corneal swelling, blurred vision, and eye infections.

### BRIEF DESCRIPTION OF THE DRAWINGS

Implementations of the present technology will now be described, by way of example only, with reference to the attached figures.

FIG. 1 is a flowchart of an exemplary embodiment of a method for making a contact lens for detecting glucose level in tears.

FIG. 2 is a diagram of a mold comprising a female mold portion and a male mold portion used in the method of FIG. 1.

FIG. 3 is a diagram showing a biosensor placed in the female mold portion of FIG. 2.

FIG. 4 is a diagram showing a gel precursor added in the female mold portion of FIG. 3.

FIG. 5 is a diagram showing the female mold portion and the male mold portion covered and exposed to ultraviolet radiation.

FIG. 6 is a diagram of the contact lens formed after the ultraviolet radiation of FIG. 5.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to illustrate details and features of the present disclosure better.

The disclosure is illustrated by way of example and not by way of limitation in the figures of the accompanying drawings, in which like references indicate similar elements. It should be noted that references to “an” or “one” embodiment in this disclosure are not necessarily to the same embodiment, and such references mean at least one.

The term “comprising,” when utilized, means “including, but not necessarily limited to”; it specifically indicates open-ended inclusion or membership in the so-described combination, group, series, and the like.

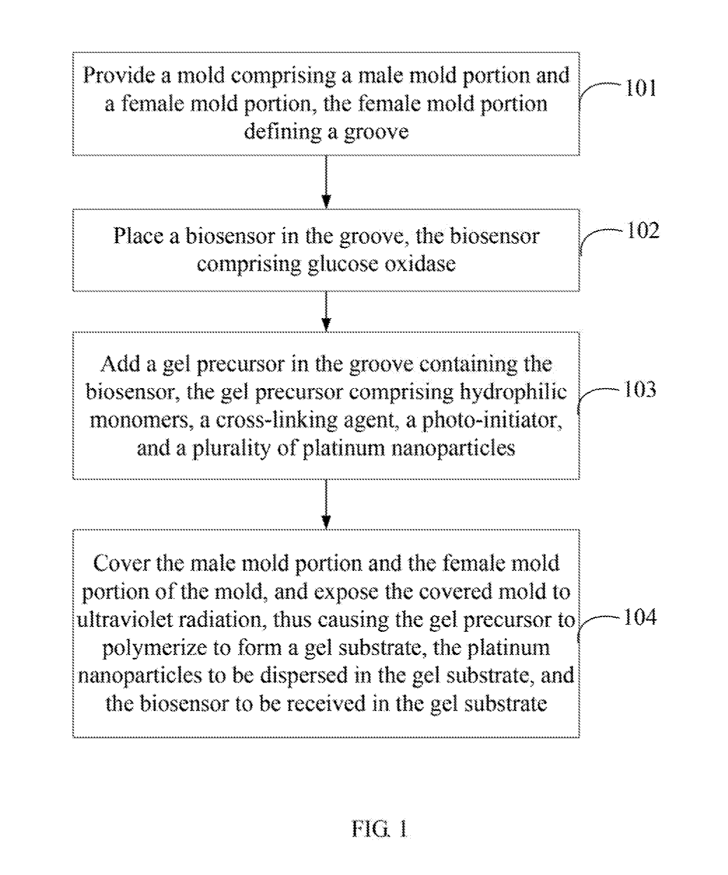

FIG. 1 illustrates a flowchart of an embodiment for a method for making a contact lens for detecting glucose level in tears. The exemplary method is provided by way of example, as there are a variety of ways to carry out the method. Each block shown in the figure represents one or more processes, methods, or subroutines, carried out in the exemplary method. Furthermore, the illustrated order of blocks is by example only, and the order of the blocks can change. Additional blocks may be added or fewer blocks may be utilized, without departing from this disclosure. The exemplary method can begin at block 101.

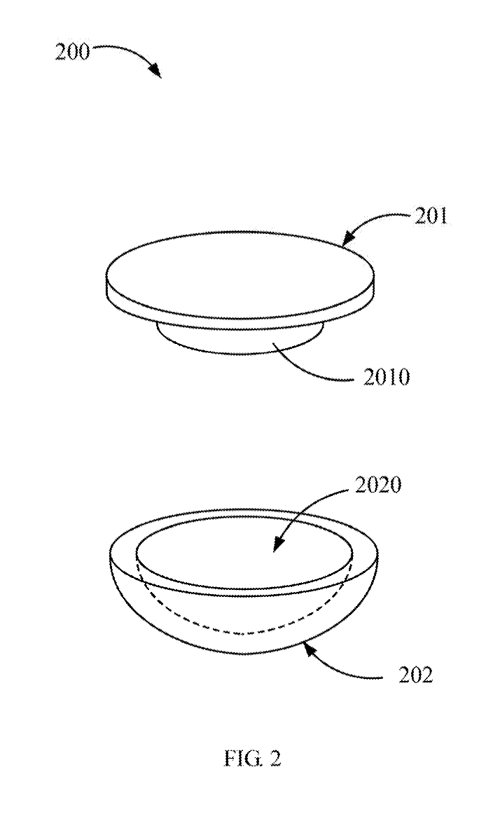

At block 101, referring to FIG. 2, a mold 200 is provided that comprises a male mold portion 201 and a female mold portion 202. The female mold portion 202 defines a groove 2020. The male mold portion 201 comprises a protruding block 2010. When the male mold portion 201 covers the female mold portion 202, the protruding block 2010 and the groove 2020 cooperatively define a molding space (not shown) which matches the contact lens 100 in shape.

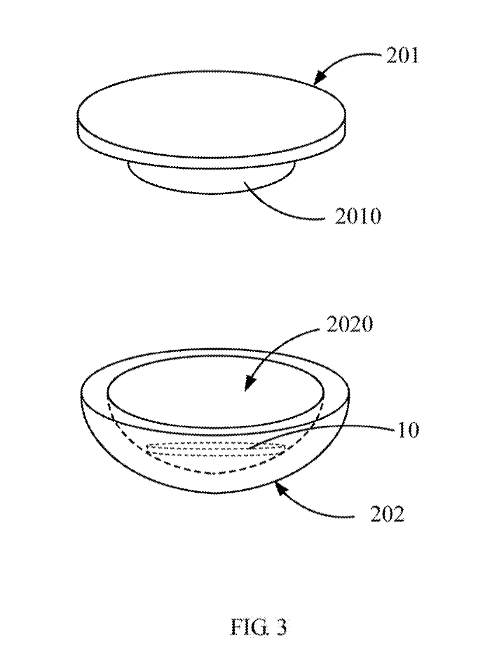

At block 102, referring to FIG. 3, a biosensor 10 is placed in the groove 2020. The biosensor 10 can detect and indicate glucose level in tears.

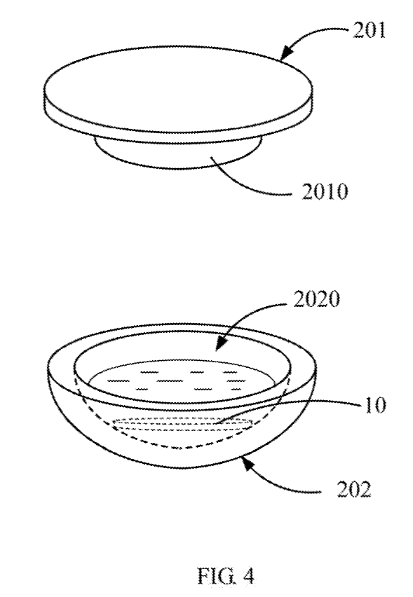

At block 103, referring to FIG. 4, a gel precursor is added in the groove 2020 containing the biosensor 10. The gel precursor comprises hydrophilic monomers, a cross-linking agent, a photo-initiator, and a plurality of platinum (Pt) nanoparticles. Each of the platinum nanoparticles has a diameter of about 5 nm to about 200 nm.

In at least one exemplary embodiment, the hydrophilic monomers have a mass percentage of about 68.4% to about 99.2% of a total mass of the gel precursor. The cross-linking agent has a mass percentage of about 0.03% to about 14.2% of the total mass of the gel precursor. The photo-initiator has a mass percentage of about 0.12% to about 18.6% of the total mass of the gel precursor. The platinum nanoparticles have a mass percentage of about 0.001% to about 0.2% of the total mass of the gel precursor.

In at least one exemplary embodiment, the hydrophilic monomers can be selected from a group consisting of 2-hydroxyethyl methacrylate (HEMA), N,N′-dimethylacrylamide (DMA), methyl methacrylate (MMA), N-vinyl pyrrolidone (NVP), polyethylene glycol maleate (PEGMA), 3-[tris(trimethylsilyl)silyl]propylmethacrylate (TRIS), polydimethylsiloxane (PDMS), hydroxyethyl acrylate (HEA), hydroxypropyl methacrylate (HPMA), dimethylaminoethyl methacrylate (DMAEMA), methyl acrylate (MA), and any combination thereof.

The cross-linking agent can be selected from a group consisting of ethylene glycol dimethacrylate (EGDMA), trimethylolpropane trimethacrylate (TMPTMA), N,N′-methylenediacrylamide (MBAA), and any combination thereof.

The photo-initiator can be selected from a group consisting of benzoin methyl ether, diethoxyacetophenone, benzoylphosphine oxide initiator, 1-hydroxycyclohexyl phenyl ketone, Darocure type initiator, Irgacure type initiator, and any combination thereof. The benzoylphosphine oxide initiator may be selected from a group consisting of 2,4,6-trimethylbenzoyldiphenylophosphine oxide, bis-(2,6-dichlorobenzoyl)-4-N-propylphenylphosphine oxide, and bis-(2,6-dichlorobenzoyl)-4-N-butylphenylphosphine oxide. The Irgacure type initiator can be Irgacure-1173 (the trade name, available commercially from Chemical Industries Basel Corporation as a clear liquid).

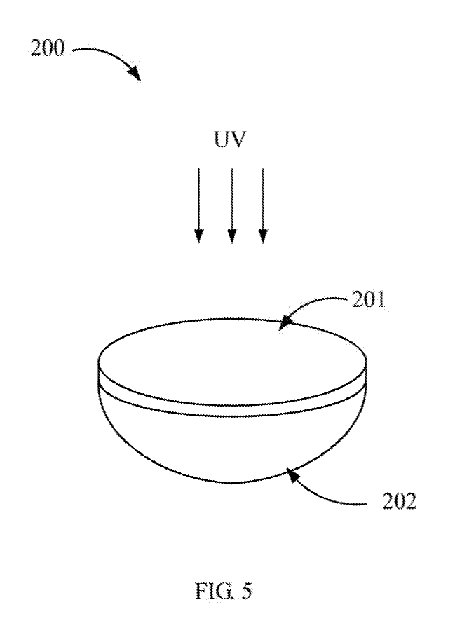

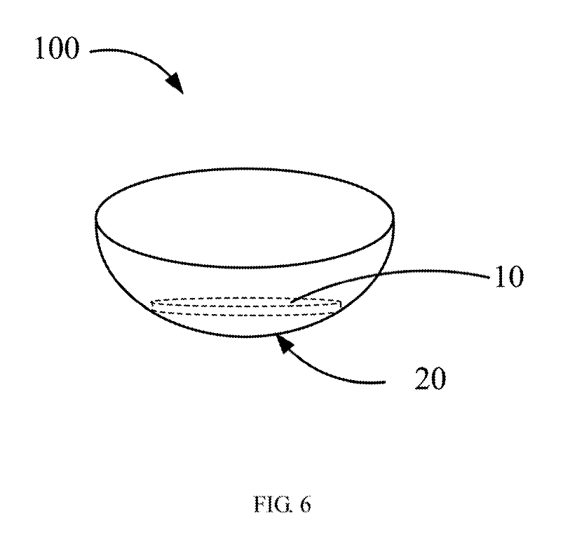

At block 104, referring to FIGS. 5 and 6, the male mold portion 201 of the mold 200 covers the female mold portion 202 to define the molding space, and the covered mold 200 is exposed to ultraviolet radiation. The gel precursor polymerizes in the molding space under the ultraviolet radiation to form a gel substrate 20. The platinum nanoparticles are dispersed in the gel substrate 20, and the biosensor 10 received in the gel substrate 10. Thus, the contact lens 100 is formed.

The biosensor 10 comprises an electrode (not shown) that comprises glucose oxidase (GOx). When the biosensor 10 contacts glucose in tears, since the gel substrate 20 is microscopically porous, the glucose can enter the gel substrate 20 and react with oxygen in the ambient environment under the function of glucose oxidase, to form glucose acid and hydrogen peroxide (H₂O₂). The hydrogen peroxide can degrade to form water and oxygen. The glucose level in tears is in proportion to the concentration of the hydrogen peroxide, and the concentration of the hydrogen peroxide is in proportion to the concentration of oxygen. Thus, the glucose level in tears can be measured by determining the concentration of oxygen. Furthermore, the platinum nanoparticles can function as a catalytic agent that can accelerate the degradation of the hydrogen peroxide, namely, accelerate the generation of water and oxygen. The reaction between the glucose and the biosensor 10 is as follows:

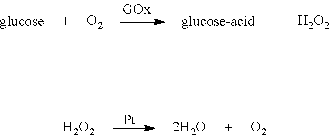

### Example 1

A biosensor 10 was placed in the groove 2020 of a female mold portion 202. A gel precursor was added in the groove 2020. The gel precursor comprised HEMA, EGDMA, Irgacure1173, and a plurality of platinum nanoparticles. In the gel precursor, the HEMA had a mass percentage of 98%, the EGDMA had a mass percentage of 0.82%, the Irgacure1173 had a mass percentage of 1.06%, the platinum nanoparticles had a mass percentage of 0.12%, and each platinum nanoparticle had a diameter of 52 nm. The male mold portion 201 covered the female mold portion 202, and the covered mold 200 was exposed to ultraviolet radiation of 365 nm for 12 minutes, thereby forming the contact lens 100.

### Example 2

A biosensor 10 was placed in the groove 2020 of a female mold portion 202. A gel precursor was added in the groove 2020. The gel precursor comprised HEMA, NVP, TRIS, TMPTMA, Irgacure1173, and a plurality of platinum nanoparticles. In the gel precursor, the HEMA had a mass percentage of 25.1%, the NVP had a mass percentage of 42.6%, the TRIS had a mass percentage of 30.45%, the TMPTMA had a mass percentage of 0.87%, the Irgacure1173 had a mass percentage of 0.92%, the platinum nanoparticles had a mass percentage of 0.06%, and each platinum nanoparticle had a diameter of 105 nm. The male mold portion 201 covered the female mold portion 202, and the covered mold 200 was exposed to ultraviolet radiation of 365 nm for 23 minutes, thereby forming the contact lens 100.

FIG. 6 illustrates an exemplary embodiment of a contact lens 100. The contact lens 100 comprises a gel substrate 20 and a number of platinum nanoparticles dispersed in the gel substrate 20. Each of the platinum nanoparticles has a diameter of about 5 nm to about 200 nm. The platinum nanoparticles have a mass percentage of about 0.001% to about 0.2% of the total mass of the gel substrate 10.

The gel substrate 20 further comprises a biosensor 10 received therein. The biosensor 10 comprises glucose oxidase which can detect glucose level in tears.

In at least one exemplary embodiment, the gel substrate 20 is made of hydrogel or silicone hydrogel.

With the above configuration, the contact lens 100 comprises the biosensor 10 for detecting glucose level in tears. Furthermore, the platinum nanoparticles dispersed in the gel substrate 20 accelerate the generation of water and oxygen, thus the contact lens 100 can prevent the user from suffering from hypoxia.

Depending on the embodiment, certain of the steps of methods hereinbefore described may be removed, others may be added, and the sequence of steps may be altered. It is also to be understood that the description and the claims drawn to a method may include some indication in reference to certain steps. However, the indication used is only to be viewed for identification purposes and not as a suggestion as to an order for the steps.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. A method for making a contact lens for detecting glucose level in tears comprising:
providing a mold comprising a male mold portion and a female mold portion, the female mold portion defining a groove;
placing a biosensor in the groove, the biosensor comprising glucose oxidase;
adding a gel precursor in the groove containing the biosensor, the gel precursor comprising hydrophilic monomers, a cross-linking agent, a photo-initiator, and a plurality of platinum nanoparticles; and
covering the male mold portion with the female mold portion; and
exposing the covered mold to ultraviolet radiation.

2. The method of claim 1, further comprising, causing the gel precursor to polymerize to form a gel substrate, dispersing the platinum nanoparticles in the gel substrate, and receiving the biosensor in the gel substrate, thereby forming the contact lens.

3. The method of claim 1, wherein each of the plurality of platinum nanoparticles has a diameter of about 5 nm to about 200 nm.

4. The method of claim 1, wherein the hydrophilic monomers have a mass percentage of about 68.4% to about 99.2% of a total mass of the gel precursor, the cross-linking agent has a mass percentage of about 0.03% to about 14.2% of the total mass of the gel precursor, the photo-initiator has a mass percentage of about 0.12% to about 18.6% of the total mass of the gel precursor, and the plurality of platinum nanoparticles has a mass percentage of about 0.001% to about 0.2% of the total mass of the gel precursor.

5. The method of claim 1, wherein the hydrophilic monomers are selected from a group consisting of 2-hydroxyethyl methacrylate, N,N′-dimethylacrylamide, methyl methacrylate, N-vinyl pyrrolidone, polyethylene glycol maleate, 3-[tris(trimethylsilyl)silyl]propylmethacrylate, polydimethylsiloxane, hydroxyethyl acrylate, hydroxypropyl methacrylate, dimethylaminoethyl methacrylate, methyl acrylate, and any combination thereof.

6. The method of claim 1, wherein the cross-linking agent is selected from a group consisting of ethylene glycol dimethacrylate, trimethylolpropane trimethacrylate, N,N′-methylenediacrylamide, and any combination thereof.

7. The method of claim 1, wherein the photo-initiator is selected from a group consisting of benzoin methyl ether, diethoxyacetophenone, benzoylphosphine oxide initiator, 1-hydroxycyclohexyl phenyl ketone, Darocure type initiator, Irgacure type initiator, and any combination thereof.

8. The method of claim 7, wherein the benzoylphosphine oxide initiator is selected from a group consisting of 2,4,6-trimethylbenzoyldiphenylophosphine oxide, bis-(2,6-dichlorobenzoyl)-4-N-propylphenylphosphine oxide, and bis-(2,6-dichlorobenzoyl)-4-N-butylphenylphosphine oxide.

9. A contact lens for detecting glucose level in tears comprising:
a gel substrate;
a plurality of platinum nanoparticles dispersed in the gel substrate; and
a biosensor received in the gel substrate, the biosensor comprising glucose oxidase.

10. The contact lens of claim 9, wherein each of the plurality of platinum nanoparticles has a diameter of about 5 nm to about 200 nm.

11. The contact lens of claim 9, wherein the plurality of platinum nanoparticles has a mass percentage of about 0.001% to about 0.2% of the total mass of the gel substrate.

12. The contact lens of claim 9, wherein the gel substrate is made of hydrogel or silicone hydrogel.
